(19) United States
(12) Patent Application Publication
(10) Pub. No.: US 2005/0265516 A1
(43) Pub. Date: Dec. 1, 2005

(54) Method and device for increasing patient safety in clinical scanners

(76) Inventor: Sultan Haider, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/126,789

(22) Filed: May 11, 2005

(30) Foreign Application Priority Data
May 14, 2004 (DE) 10 2004 024 097.3

(51) Int. Cl. A61B 6/00; G01N 23/00

(52) U.S. Cl. 378/20

(57) ABSTRACT

In a method and device for increasing patient safety in clinical scanners, the positioning of the patient during the preparation and the implementation of the examination is monitored by a 3D camera system, and incorrect positions are automatically detected by comparison of the current image with standard specifications, and corresponding corrections are prompted.

### BACKGROUND OF THE INVENTION

1. Field of the Invention

The present invention concerns a method and device for optimization of the use of clinical scanners with increased patient safety

2. Description of the Prior Art

Achieving an optimized arrangement of a patient in a clinical scanner, for example an MR scanner, is of decisive importance for the success of an examination. For example, it depends on whether the patient is inserted into the scanner in the correct bearing and in the correct position (head first or feet first). Given the absence of an optimal alignment is possible, the patient must be moved out again and be positioned differently, which means a significant time expenditure that—in view of the cost of approximately $30 per operational minute of such a scanner—detracts significantly from the “bottom line” and can be decisive for the cost-effectiveness of the operation of such a scanner.

It is even more decisive that, for many examinations, the patient must lie in the scanner such that overlapping of body parts and/or contact between body parts is prevented, to avoid electrical induction loops.

### SUMMARY OF THE INVENTION

An object of the present invention is to provide a method that ensures optimal positions of a patient in an examination apparatus without the overly involving of monitoring personnel, and that allows the optimal position to be continuously maintained during the entire process of an examination.

This object is achieved in accordance with the invention by a method wherein the positioning of the patient is monitored by a 3D camera system during the preparation and the implementation of the examination, and improper positions are automatically detected by comparison with specifications (targets) and corresponding corrections are provided.

In this manner positions of the patient are automatically detected in which extremities contact one another or with the body trunk and form electrical current loops.

Such an automatic monitoring is important because it is not sufficient that operating personnel of the scanner correctly position the patient at the beginning with the extremities properly arranged so that the cited contacts are prevented. Because such examinations can frequently be of long duration, it cannot be precluded that the patient (for whom a long-duration, extremely immobile posture is exceedingly uncomfortable) may move after being correctly positioned so that the contacts forming current loops subsequently occur. Such movements often are not detected within the narrow scanner tube by the operating personnel, with the consequence of (possibly drastic) adverse health effects on the patient due to current surges.

For implementation of the method, a 3D image camera system is disposed in front of and/or within a scanner and is connected with an evaluation and monitoring device containing stored standard positions, preferably integrated into the scanner operating system.

### DESCRIPTION OF THE DRAWINGS

FIG. 1 is a schematic, perspective view of a scanner with a patient arranged in front thereof.

FIG. 2 is a schematic illustration of the camera system and the associated evaluation device in accordance with the invention.

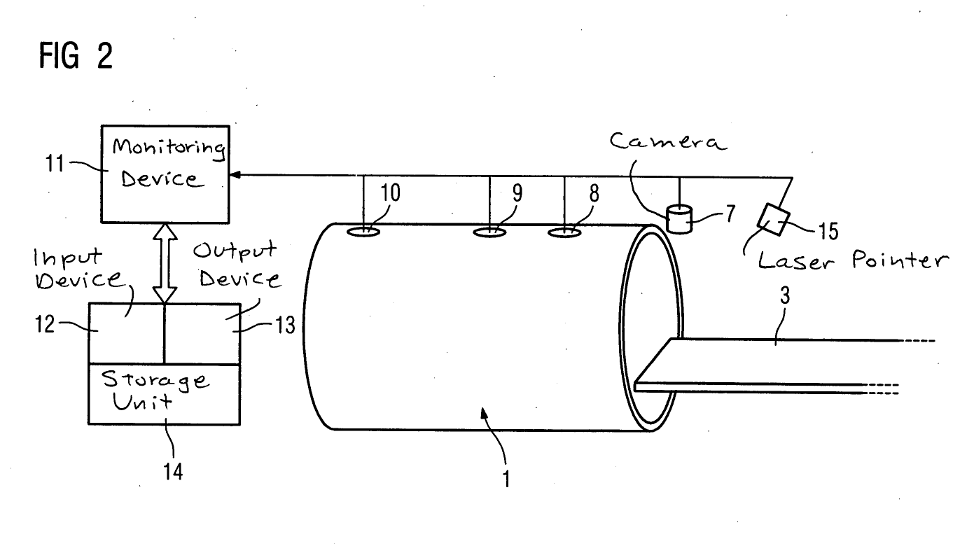

FIG. 3 illustrates a number of possible incorrect positions of the patient that are automatically recognized by the inventive system and are remedied by appropriate re-arrangement of the patient.

### DESCRIPTION OF THE PREFERRED EMBODIMENTS

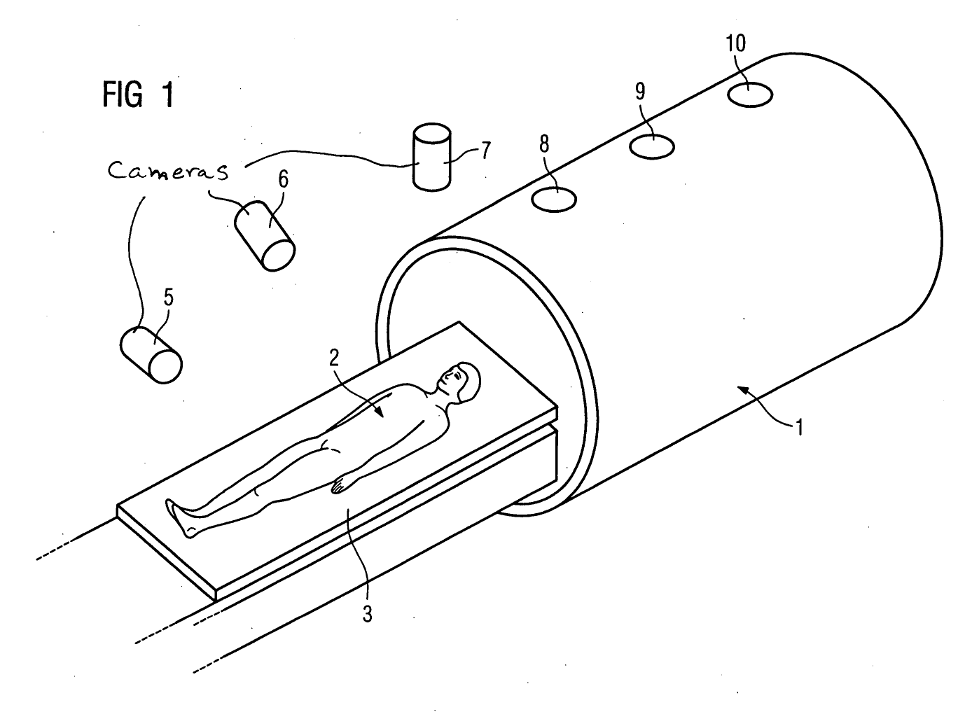

In FIG. 1, a scanner tube 1 can be seen (for example for an MR scanner) into which a patient 2 can be inserted on a movable patient bed 3.

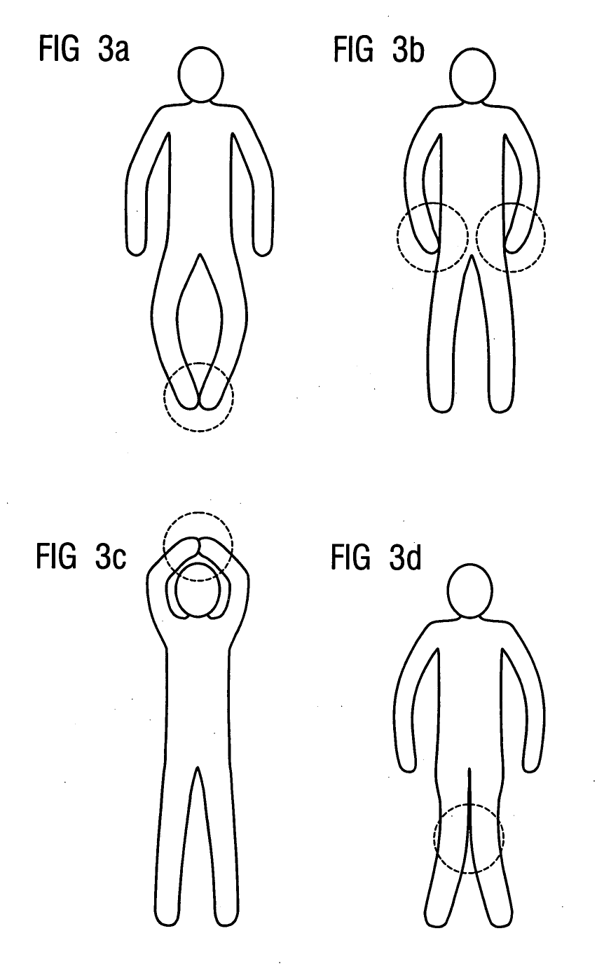

Some cameras for a 3D image system are shown at 5, 6 and 7, with which observation of the patient can ensue outside of the scanner tube 1. A 3D image system for monitoring the interior of the scanner tune 1 also can be formed by cameras that can be introduced into the interior through openings 8, 9 and 10. Further cameras at the head end are directed inwardly. Incorrect positioning of the patient (as shown in FIGS. 3a through 3d) are detected by this camera system and corrected by a suitable re-arrangement of the patient. These are positions in which extremities contact one another or contact the body trunk to form electrical current loops, such that pain or even serious adverse health effects could occur in the examination due to current flow in the patient's body. The 3D image camera system can identify the acquired images in terms of their importance by algorithms entered into the evaluation and monitoring device 11 and standard positions stored therein. The illustrated laterally-detected camera position 5 of the patient is of importance in order, for example, to be able to differentiate desired positions in which the patient holds his or her hands at an angle over the body from a position similar to FIG. 3b, in which the hands lie against the body and then form current loops to be avoided. The apparatus has an input device 12 and an output device 13 of the evaluation and monitoring device 11 with connected data storage unit 14. As soon as the evaluation and monitoring device 11 has detected an incorrect position, for example according to one shown in FIGS. 3a through 3d, a corresponding, preferably acoustic indication automatically ensues to the operating personnel or the patient via a speaker arranged in the scanner. The instruction requests a correction of the incorrect position. At the same time monitoring naturally continues, and thus it can also be absolutely ensured that the incorrect positions to be avoided are corrected with certainty immediately given a temporary occurrence. A projector with a laser pointer is shown at 15 that marks the body region in which a variation occurs due to movement of body parts, the axis of the projector being controlled by the monitoring system 11, and therewith via the camera system.

Although modifications and changes may be suggested by those skilled in the art, it is the intention of the inventor to embody within the patent warranted hereon all changes and modifications as reasonably and properly come within the scope of his contribution to the art.

1. A method for optimizing patient safety during acquisition of data from a subject in a data acquisition unit, comprising the steps of:
positioning a patient in a data acquisition unit;
during acquisition of data from the patient with said data acquisition unit, monitoring a position of the patient in said data acquisition unit with a 3D camera system; and
automatically electronically detecting an incorrect position of the patient during the data acquisition by comparing the position of the patient acquired by said 3D camera system with at least one stored position, and prompting a correction of the position of the patient if an incorrect position is detected.

2. A method as claimed in claim 1 comprising storing, as an incorrect position, at least one position of the patient selected from the group of positions consisting of a position wherein an extremity of the patient contacts another extremity of the patient, and a position wherein an extremity of the patient contacts the body trunk of the patient.

3. An apparatus for acquiring imaging data from a patient, comprising:
a data acquisition unit adapted to receive a patient therein to acquire data therefrom;
a 3D camera system mounted relative to said data acquisition unit to monitor, during acquisition of data from the patient with said data acquisition unit, a position of the patient in said data acquisition unit and generate position data; and
a computer supplied with said position data to automatically electronically detect an incorrect position of the patient during the data acquisition by comparing said position data of the patient acquired by said 3D camera system with at least one set of stored position data, and prompting a correction of the position of the patient if an incorrect position is detected.

4. An apparatus as claimed in claim 3 comprising a laser pointer, automatically controlled by said computer, to optically designate a point at the body of the patient in the scanner where said incorrect position exists.
